SEQUENCE LISTING (14 sequences)

SEQ ID NO: 1 - protein, 8 aa, ARTIFICIAL SEQUENCE
  Synthetic polypeptide
KSPKKAKK

SEQ ID NO: 2 - protein, 7 aa, ARTIFICIAL SEQUENCE
  Synthetic polypeptide
FYPWMKG

SEQ ID NO: 3 - protein, 7 aa, ARTIFICIAL SEQUENCE
  Synthetic polypeptide
FYPAAKG

SEQ ID NO: 4 - DNA, 72 bp, ARTIFICIAL SEQUENCE
  Eco-RI-PvuII insert
gatcgcgagctcattgggcagcgagctcattggccaagcagctcatagcttggcgtaatcatggtcatagct

SEQ ID NO: 5 - DNA, 47 bp, ARTIFICIAL SEQUENCE
  Synthetic oligonucleotide
gatctcccggcgaatgattgaccatatcggcgccactgtcacccgga

SEQ ID NO: 6 - DNA, 40 bp, ARTIFICIAL SEQUENCE
  Synthetic oligonucleotide
gatctcccggcgaggattcttgaccatatcggcgccaccc

SEQ ID NO: 7 - DNA, 40 bp, ARTIFICIAL SEQUENCE
  Synthetic oligonucleotide
gatctcccggcgaggatgatttaacatatcggcgccaccc

SEQ ID NO: 8 - DNA, 47 bp, ARTIFICIAL SEQUENCE
  Synthetic oligonucleotide
gatctcccggcgaatgatttatggtatcggcgccactgtcacccgga

SEQ ID NO: 9 - protein, 9 aa, ARTIFICIAL SEQUENCE
  Synthetic polypeptide
YPWMIFDWM

SEQ ID NO: 10 - protein, 4 aa, ARTIFICIAL SEQUENCE
  Synthetic polypeptide
YPWM

SEQ ID NO: 11 - protein, 4 aa, ARTIFICIAL SEQUENCE
  Synthetic polypeptide
YKWM

SEQ ID NO: 12 - protein, 4 aa, ARTIFICIAL SEQUENCE
  Synthetic polypeptide
FDWM

SEQ ID NO: 13 - protein, 5 aa, ARTIFICIAL SEQUENCE
  Synthetic polypeptide
FYPWM

SEQ ID NO: 14 - protein, 6 aa, ARTIFICIAL SEQUENCE
  Synthetic polypeptide
FYPAAK

(19) United States
(12) Patent Application Publication
(10) Pub. No.: US 2005/0123936 A1
(43) Pub. Date: Jun. 9, 2005

(54) Method, composition, and kit to design, evaluate, and/or test compounds that modulate regulatory factor binding to nucleic acids

(76) Inventor: Aseem Z. Ansari, Madison, WI (US)

Correspondence Address: Intellectual Property Department;DEWITT ROSS & STEVENS S.C., US Bank Building, 8000 Excelsior Drive Suite 401, Madison, WI, 53717-1914, US

(21) Appl. No.: 10/759,416

(22) Filed: Jan. 16, 2004

Related U.S. Application Data
(60) Provisional application No. 60/440,494, filed on Jan. 16, 2003.

(51) Int. Cl. C12Q 1/68

(52) U.S. Cl. 435/6

(57) ABSTRACT

A method of evaluating one or more test compounds to identify test compounds that modulate binding of natural or artificial regulatory factors to corresponding single-, double-, or triple-stranded nucleic acid binding sites is described. The method utilizes an isolated nucleic acid target that defines at least one known or putative binding site for a regulatory factor. The nucleic acid target has conjugated or covalently bonded thereto, at a point proximate to, but not within, the binding site: (i) an anchor moiety; (ii) a linker moiety bonded to the anchor moiety; and (iii) a test compound bonded to the linker moiety. To evaluate the test compound, the nucleic acid target of step is then contacted to a reagent mixture comprising one or more natural or artificial regulatory factors specific for the binding site defined in the nucleic acid target. It is then determined, by any number of known methods, whether binding of the regulatory factor to the binding site defined in the nucleic acid target was modulated by presence of the test compound.

### CROSS-REFERENCE TO RELATED APPLICATIONS

Priority is hereby claimed to provisional application Ser. No. 60/440,494, filed Jan. 16, 2003, the content of which is incorporated herein by reference.

### BACKGROUND

1. Overview:

All cells in an organism, with a few exceptions, bear the same genome. Yet cells specialize to yield tissues having diverse morphology and function. This diversity arises due to the differences in sets of genes that are expressed in a programmed manner during development and cellular differentiation. The recent decoding of the human genome, coupled with genome-wide expression profiling, is clarifying the relationship between specific gene expression patterns and ultimate cellular fates. Gene expression patterns are controlled by a host of transcription regulatory factors. For a full discussion of the present state of the art regarding transcription factors, see, for example, Ptashne & Gann (2001) “Genes & Signals,” Cold Spring Harbor Laboratory Press, Cold Spring Harbor, N.Y. In diseases as disparate as diabetes and cancer, it is often one or more malfunctioning transcription regulatory factors that produce the aberrant patterns of gene expression that are at the heart of the ailment. For specific examples, see Perou et al. (2000) Nature, 406:747-752; Duncan et al. (1998) Science, 281:692-695; and Pandolfi (2001) Oncogene, 20:3116-3127.

In this context, rationally developing synthetic molecules designed to control the expression of specific genes is an important venue for study. See, for example, Ansari (2001) Curr. Org Chem. 5:903-921. Ideally, such artificial transcription factors (“ATFs”) are designed from the outset to regulate (either positively or negatively) any gene or any set of genes without influencing the expression of other genes in the genome. Alternatively, compounds that can be designed to modulate in vivo the functionality of endogenous transcription factors are also an important venue for study. In like fashion, these compounds, designated transcription factor modulators (“TFMs”), or more generically designated regulatory factor modulators (“RFMs”) can be designed to regulate, either positively or negatively, a pre-selected gene or set of genes.

Such molecules (ATFs, TFMs, and RFMs) would serve as powerful tools for functional genomics, as well as for unraveling key transcriptional events involving individual genes (events that perhaps govern cell fate). In short, compounds that exert their pharmacological activity via modulating the interaction of regulatory factors with their corresponding regulatory factor binding sites have significant therapeutic potential.

The nascent field of ATF design also holds tremendous promise toward generating molecules that would help elucidate subtle mechanistic features of transcriptional regulation. ATFs can also serve as tools to dissect regulatory decisions that govern cellular differentiation or disease.

There exists, however, a long-felt and unmet need for a robust means by which putative ATFs, TFMs, and RFMs can be identified and characterized. The present invention is drawn to methods and compositions of matter to aid in this effort.

2. Sequence-Specific Recognition of Nucleic Acids:

Sequence-specific recognition of DNA by regulatory factors, such as transcription factors and repressors plays a central role in cellular gene regulation at the transcriptional level. Such recognition and is also crucial in both DNA replication and recombination. As noted in the previous paragraph, one long-term (and unmet) objective of structural and biophysical studies of protein-nucleic acid interactions is to characterize the molecular basis for these processes. Elucidating the nature of these protein-nucleic acid interactions is highly useful because it provides valuable information for: 1) designing drugs that modulate gene expression by altering the reaction between an endogenous transcription factor and its corresponding nucleic acid binding site; and/or 2) designing ATFs that can compete for binding at a nucleic acid binding site that is relevant to a given disease state.

Protein-nucleic acid recognition is regulated at the molecular level by a combination of hydrogen bonds, electrostatic interactions, and van der Waals contacts between individual amino acid residues of the protein and selected nucleotides in the nucleic acid target. As of January 2004, however, no sequence-specific recognition code (based upon the nucleotide sequence of the target, the amino acid sequence of the transcription factor, or a combination thereof) has been identified. Thus, the sequence specificity of protein-nucleic acid binding apparently cannot be reduced to a canonic relationship like the three-letter nucleotide codons that correlate a gene's nucleotide sequence to the amino acid sequence of the encoded polypeptide. In contrast, the current state-of-the-art suggests that the entire binding region of the protein exhibits a certain molecular complementarity to the major or minor groove of the nucleic acid target. See Bewley et al. (1998) Annu. Rev. Biophys. Biomol. Struct., 27:105-131.

That being said, there has been some progress in developing small, synthetic ligands that recognize DNA on a sequence-specific (or at least sequence-preferred) basis. These compounds generally fall into one of several loosely-defined groups of compounds: major-groove-binding/triple helix-forming oligonucleotides, helix-invading peptide nucleic acids (“PNAs”), and minor groove-binding polyamides (“PAs”). See, for example, Fox (2000) Curr. Med. Chem. 7:17-37; Nielsen (2001) Methods Enzymol., 340:329-340; and Dervan & Burli (1999) Curr. Opin. Chem. Biol., 3:688-693, respectively. So-called “zinc-finger” peptides have also been constructed to target proteins to nucleic acids in a three-nucleotide, sequence-specific fashion. See Choo & Isalan (2000) Curr. Opin. Struct. Biol. 10:411-416. Several attempts have been made to identify small peptides that bind double-stranded DNA sequence-specifically, but these attempts have proven unsuccessful. See, for example, Behrens & Nielsen (1998) Combin. Chem. High Throughput Screening 1:127-134; and Chang & Herdewijn (2001) Curr. Med. Chem. 8:517-531.

There are a number of conventional fluorophores that are known to bind to double-stranded DNA with a preference for a specific type of nucleotide sequence motif For example, the fluorophore Hoechst 33258 is a conventional DNA-binding agent consisting of two linked benzimidazole moieties, a phenol moiety at one terminus, and an N-methlypiperazine moiety at the other terminus. Hoechst 33258 has a very pronounced binding preference for AT-rich regions. The interaction between Hoechst 33258 and double-stranded DNA has been characterized extensively using DNase I footprinting, electric linear dichroism, solution-phase NMR techniques, and X-ray crystallography. These studies reveal that Hoechst 33258 binds with high affinity to the minor groove of double stranded B-DNA, with a strong preference for AT-rich regions.

Behrens et al. (2001) Bioconjugate Chem, 12:1021-1027 have incorporated analogs of Hoechst 33258 onto the N-terminus of a defined polypeptide backbone to yield a polypeptide-containing compound the retains the AT-rich binding preference of the unmodified Hoechst 33258 dye. In this work, a Hoechst 33258 analog was bonded to the N-terminus of the cationic polypeptide KSPKKAKK (SEQ. ID. NO: 1). The cationic polypeptide so modified was found to bind to double-stranded DNA with approximately 10-fold higher affinity than the Hoechst analog itself, without altering the AT-rich binding preference of the unmodified Hoechst 33258 dye.

3. Sequence-Specific Polyamides:

Known in the prior art are polyamides (PAs) containing N-methylpyrrole (Py) and N-methylimidazole (Im) amino acids that are capable of binding to duplex DNA on a sequence-specific (or sequence-preferred) fashion. For side-by-side complexes of Py/Im-PA's, where the PA binds in the minor groove of the DNA, the sequence specificity depends on the sequence of side-by-side amino acid pairings in the PA. See Wade et al. (1992) J. Am. Chem. Soc., 114:8783-8794; Mrksich et al. (1992) Proc. Natl. Acad. Sci. U.S.A., 89:7586-7590; and Wade et al. (1993) Biochemistry 32:11385-11389. A pairing of Im opposite Py targets a G•C base pair while a pairing of Py opposite Im targets a C•G base pair. A Py/Py combination is degenerate, targeting both A•T and T•A base pairs. Specificity for G•C base pairs it thought to result from the formation of a putative hydrogen bond between the imidazole N3 and the exocyclic amine group of guanine. The pairing rules are generally supported by a variety of footprinting and NMR structure studies. See, for example, Mrksich et al. (1993) J. Am. Chem. Soc., 115:2572; Geierstanger et al. (1994) Science, 266:646; and Mrksich et al. (1995) J. Am. Chem. Soc., 117:3325.

While PAs can be fabricated that will bind to DNA sequence-specifically, the binding affinities of PAs are generally modest when compared to the binding affinities of natural DNA binding proteins. Clemens et al. (1994) J. Mol. Biol. 244:23-25. For example DNA-binding transcription factors recognize their corresponding DNA binding sites at sub-nanomolar concentrations. Jamieson et al. (1994) Biochemistry 33:5689-5695; Choo & Klug (1994) Proc. Natl. Acad. Sci. U.S.A. 91:11168-11172; and Greisman & Pabo (1997) Science 275:657-661. As a general rule, six-ring hairpin polyamides require concentrations on the order of 10 nM to occupy their target sites.

4. Synthetic Transcription Antagonists:

Two approaches for developing synthetic transcriptional antagonists have been described in the prior art: triple-helix forming compounds and cell-permeable carbohydrate compounds. Oligodeoxynucleotides that recognize the major groove of duplex DNA and bind thereto via triple helix formation have a broad sequence repertoire, high affinity, and high specificity. See Moser & Dervan (1987) Science 238:645-650; and Thuong et al. (1993) Agnew. Chem. Int. Ed. Engl. 32:666-690. On one hand, triplex-forming oligonucleotides and their analogs have been shown to interfere with gene expression, see Maher et al. (1992) Biochemistry 31:70-81; and Duvalvalentin et al. (1992) Proc. Natl. Acad. Sci. U.S.A. 89:504-508. On the other hand, the triple helix approach is limited to purine tracks and suffers from poor cellular uptake.

There are also a few examples of cell-permeable carbohydrate based ligands that interfere with transcription factor function. See Ho (1994) Proc. Natl. Acad. Sci. USA, 91:9203-9207; and Liu et al. (1996) Proc. Natl. Acad. Sci. USA, 93:940-944.

### SUMMARY OF THE INVENTION

Key principals in the regulation of transcription include, on one hand, the identification of activators that bind sequence-specific binding sites on genomic DNA and by mechanisms (still unclear) recruit the required transcriptional machinery and chromatin remodeling or modifying enzymes. On the other hand, repressors of transcription mask or displace activators from promoters, dismantle the transcriptional machinery and/or recruit chromatin-condensing or modifying enzymes and co-factors to the DNA. Despite a vast amount of research into the mechanisms of transcription, there remains significant controversy regarding the mechanism by which activators or repressors exert their function. In short, it is one matter to address whether a given compound modulates expression of a gene (either by increasing expression or decreasing expression). It is another matter entirely to address how the transcriptional machinery effects and regulates transcription, how a given compound interferes with that mechanism, and how to quantify the modulation.

Several key issues regarding regulatory factors that have yet to be resolved include the structures of activating domains and whether those structures are conserved, the sequence-specific binding site to which the regulatory factors bind, their mode of activation, and the role played by chromatin-bound DNA. The prior art, briefly discussed above, presents several seemingly incompatible theories. For example, in the case of structure, certain activating peptides are thought to exist as “acid blobs.” However, these same activating peptides have also been theorized to adopt a clear helical structure upon binding to specific targets. Evidence that appears to support either theory in vivo confounds the issue considerably. Compare, for example, Sigler (1988) “Acid Blobs and Negative Noodles,” Nature, 333:210-212, to Uesugi et al. (1997) “Induced Alpha Helix in the VP16 Activation Domain Upon Binding to a Human TAF,” Science, 277:1310-1313. Similarly, identifying the targets of activators has also been a controversial area, with seemingly as many putative targets as there are researchers seeking them.

The present invention thus provides methods, corresponding compositions of matter, and corresponding kits to design, evaluate, and/or test compounds that modulate regulatory factor binding to nucleic acids. The approach of the method is not to measure transcription per se (or some other biological phenomenon involving nucleic acid), but rather to examine closely the binding of a given regulatory factor to its cognate nucleic acid bind site and to insert into the reaction, at a location proximate to where the regulator factor binds, a test compound that is physically linked to the nucleic acid target, but is not situated within the regulatory factor binding site. In this fashion, the test compound cannot diffuse from, or otherwise be physically displaced entirely from the local domain of the reaction being studied. In short, the test compound is physically anchored to the nucleic acid target, at a point near to the regulatory factor binding site, but not so close as to disturb the normal function of the binding site.

Thus, the preferred embodiment of the invention is directed to an in vitro method of evaluating one or more test compounds to identify test compounds that modulate binding of natural or artificial regulatory factors to corresponding single-, double-, or triple-stranded nucleic acid binding sites. The method comprises first providing an isolated nucleic acid target that defines a known or putative binding site for a regulatory factor. The isolated nucleic acid target has conjugated thereto, at a point proximate to the binding site: an anchor moiety, a linker moiety covalently bonded to the anchor moiety, and a test compound conjugated to the linker moiety. Then, under physiological conditions, the nucleic acid target is contacted in vitro to a reagent mixture comprising one or more natural or artificial regulatory factors specific for the binding site defined in the nucleic acid target. It is then determined whether the binding of the regulatory factor to the binding site defined in the nucleic acid target is modulated by the presence of the test compound.

A second embodiment of the invention is a method of evaluating one or more test compounds to identify test compounds that facilitate, recruit, or stabilize binding of natural transcription factors to corresponding single-, double-, or triple-stranded transcription factor binding sites on nucleic acid. Here, the method comprises providing an isolated nucleic acid target that defines at least one desired transcription factor binding site. The nucleic acid target has covalently bonded thereto, at a point proximate to, but not within, the transcription factor binding site: an anchor moiety, a linker moiety covalently bonded to the anchor moiety, and a test compound bonded to the linker moiety. The nucleic acid target is then contacted in vitro (under transcription conditions) to a reagent mixture comprising one or more natural transcription factors specific for the transcription factor binding site defined in the nucleic acid target. It is then determined whether the test compound alters binding of the natural transcription factor to the nucleic acid target.

The inventive method can also be used to evaluate one or more test compounds to identify test compounds that facilitate, recruit, or stabilize binding of artificial transcription factors to corresponding single-, double-, or triple-stranded transcription factor binding sites on nucleic acid. In this embodiment, the method is the same as described in the previous two paragraphs, except that the test compound bonded to the linker moiety is known to modulate binding of natural transcription factors to a transcription factor binding site defined in the nucleic acid target. In short, in this embodiment, the test compound has already been shown, a priori, to have some modulating effect on natural transcription factors. This same test compound is then used to determine if it exerts a similar modulatory effect on a putative artificial transcription factor. In short, this approach is useful because it allows for evaluating a putative artificial transcription factor to see if it can form the same (or similar) interfaces with a test compound known to interface with a natural transcription factor that binds to the same recognition sequence.

In any of the embodiments described in the previous paragraphs, the isolated nucleic acid target may define but a single (i.e., one and only one) transcription factor or regulatory factor binding site per nucleic acid target. This allows for close control of the reaction conditions and simplifies interpreting the results of any given experiment. The isolated nucleic acid target may also define a plurality of regulatory factor binding or transcription factor binding sites per nucleic acid target.

The invention also encompasses a composition of matter. The composition comprising an isolated nucleic acid target that defines a desired or putative binding site for a regulatory factor, the isolated nucleic acid target having covalently bonded thereto, at a point proximate to the binding site an anchor moiety, a linker moiety covalently bonded to the anchor moiety, and a test compound conjugated to the linker moiety.

The invention also encompasses a kit for testing a compound for its ability to modulate binding of a regulatory factor to a corresponding regulatory factor binding site on a nucleic acid. The kit comprises an isolated nucleic acid target that defines a regulatory factor binding site. The isolated nucleic acid target comprises an anchor moiety covalently bonded thereto at a point proximate to the regulatory factor binding site, and a bifunctional linker moiety covalently bonded to the anchor moiety. The bifunctional linker moiety comprises a free terminus that is dimensioned and configured to be conjugated or covalently bonded to a compound to be tested. The isolated nucleic acid target is disposed in a suitable container, and instructions for use of the kit are normally included as part of the kit.

The isolated nucleic acid target can be any nucleic acid as that term is defined hereinbelow. The isolated nucleic acid target includes, without limitation, single-, double-, or triple-stranded nucleic acid, including, without limitation: DNA, RNA, PNA, homo- and hetero-duplexes and triplexes thereof, and modified forms thereof. At least a portion of the isolated nucleic acid target defines a desired binding site for one or more regulatory factors. For example, the nucleic acid target can define a transcription factor binding site, a promoter site, a repressor site, a co-factor binding site, or some other binding site. In short, the defined binding site within the nucleic acid target can be any known or putative nucleotide sequence that specifically or preferentially binds a proteinaceous or non-proteinaceous regulator factor.

The regulatory factor present in the reagent mixture can be any regulatory factor as that term is defined hereinbelow. The regulatory factor can be, for example, natural or artificial, such as a natural transcription factor or an artificial transcription factor, or any other natural or artificial regulatory factor.

The test compound, such as a putative RFM or TFM, a putative pharmacologically active agent, a polypeptide, a protein, an intercalator, a heterocyle, etc. (literally any test compound desired), is conjugated or covalently linked to the linker moiety. The linker moiety is bifunctional in that it acts as a bridge to link the anchor moiety to the test compound.

Using this approach, entire chemical libraries can be quickly scanned to identify molecules that facilitate, recruit, and/or stabilize binding of natural transcription factors to their corresponding nucleic acid binding sites. This can be done using wild-type transcription factors, or mutated (or otherwise altered) transcription factors. Moreover, the nucleic acid target described herein can be purposefully fabricated as a perfect sequence match for the transcription factor being studied, or purposefully designed to be a non-canonical match or a mutated match to thereby destabilize binding and gauge the effect of the destabilization.

In short, the purpose and utility of the invention is to identify and evaluate test compounds that mimic nucleic acid regulatory factors, and more specifically transcriptional regulators. The method enables test compounds to be identified that form molecular interfaces with natural or artificial transcription factors (and other regulatory factors). In another approach, where the test compound is known to interact with a natural transcription factor, the known test compound can be used as a means to screen putative ATF's and to measure whether a putative ATF will bind or interface cooperatively with a natural transcription factor. Evaluating the nature of such an interface is extraordinarily useful because it provides structural information needed to build sophisticated ATFs that act in concert with natural transcription factors. Ideally, these sophisticated ATFs would be capable of regulating different sets of promoters in response to different stimuli. Together these approaches provide powerful tools that can be used to study intractable mechanistic features of transcriptional regulation, to serve as tools to dissect genome-wide transcriptional networks, and to trigger desired transcriptional cascades and to divert and/or control the fate of cells. The method can also be used in stem cell or tissue culture engineering to evaluate the course of cellular or tissue development in the presence of a putative regulatory factor.

The present approach is inspired by the frequent appearance in nature of weak molecular interfaces to generate highly specific transcription factor ensembles at targeted promoters. Putative ATFs to be assayed by the subject method may function in concert with natural transcription factors to interpret combinatorial cellular signals. However, an important step toward reaching this goal is method to evaluate the specific, albeit weak, interfacial contacts that the putative ATFs have with natural transcriptional regulators. The present invention provides such a method.

Thus, for example, the present method can be used to identify small molecules or peptides that bind with high selectivity to any number of classes of transcriptional regulators. Having once been identified and their binding characteristics quantified, these molecules could then be utilized with other DNA-binding scaffolds and used in modular approach to ATF design. This approach is easily extended beyond transcriptional regulators, and can be used to identify and evaluate the nucleic acid binding characteristics (and cooperative tendencies) of a wide range of proteins that engage in DNA transactions.

### BRIEF DESCRIPTION OF THE FIGURES

FIG. 1a depicts the overall structure and insert sequence of the EcoRI/PvuII restriction fragment from the plasmid pDEH9, which was used for the DNAse 1 titration experiments in Example 4.

FIG. 1b is a quantitative DNAse 1 footprint titration experiment for compound 1 (see Example 4). Lane 1: Intact DNA. Lane 2: A-reaction. Lane 3: G-reaction. Lane 4: DNAse 1 standard. Lanes 5-17: 50 nM, 20 nM, 10 nM, 5 nM, 2 nM, 1 nM, 500 pM, 200 pM, 100 pM, 50 pM, 20 pM, 10 pM, 5 pM, respectively.

FIG. 1c is a quantitative DNAse 1 footprint titration experiment for compound 2 (see Example 4). Lane 1: Intact DNA. Lane 2: A-reaction. Lane 3: G-reaction. Lane 4: DNAse 1 standard. Lanes 5-17: 500 nM, 200 nM, 100 nM, 50 nM, 20 nM, 10 nM, 5 nM, 2 nM, 1 nM, 500 pM, 200 pM, 100 pM, 50 pM, respectively.

FIG. 1d is a quantitative DNAse 1 footprint titration experiment for compound 3 (see Example 4). Lane 1: Intact DNA. Lane 2: A-reaction. Lane 3: G-reaction. Lane 4: DNAse 1 standard. Lanes 5-17: 1 pM, 500 nM, 200 nM, 100 nM, 50 nM, 20 nM, 10 nM, 5 nM, 2 nM, 1 nM, 500 pM, 200 pM, 100 pM, respectively.

FIG. 2a depicts the optimal DNA duplex used for the electrophoretic mobility shift assay (EMSA) studies in Example 6.

FIG. 2b depicts an EMSA template having a 2-bp mismatch in the Exd binding site.

FIG. 2c depicts an EMSA template having a 2-bp mismatch in the PA binding site.

FIG. 2d depicts an EMSA template the defines a composite Ubx-Exd binding site.

FIG. 3a is a gel depicting the results of EMSA studies with polyamides 1-3. See Example 6.

FIG. 3b is another gel depicting the results of EMSA studies with polyamides 1-3. See Example 6.

### ABBREVIATIONS AND DEFINITIONS

The following abbreviations and definitions are used throughout the specification and claims. Terms not assigned a specific definition herein are to be afforded their accepted definition within the fields of chemistry, biochemistry, and/or genetics.

Alkyl=straight or branched chain alkyl groups having 1-6 carbon atoms, such as methyl, ethyl, propyl, isopropyl, n-butyl, sec-butyl, tert-butyl, pentyl, 2-pentyl, isopentyl, neopentyl, hexyl, 2-hexyl, 3-hexyl, and 3-methylpentyl. Preferred alkyl groups are methyl, ethyl, propyl, butyl, cyclopropyl or cyclopropylmethyl. “Alkene” and “alkyne” have their corresponding meanings for alkyl groups bearing one or more double or triple bonds. As used herein, the terms alkyl, alkene, and alkyne encompass both monofunctional groups (e.g. —CH₂CH₃) and/or their corresponding bifunctional groups (e.g. —CH2—CH2-), as context permits.

Aptamer=As used in the molecular biology arts, “aptamer” generally refers to a double-stranded DNA or single-stranded RNA moiety that binds to specific molecular target, such as a protein or metabolite. As used here herein, the term “aptamer” is explicitly given a broader meaning and encompasses a linker moiety (as defined herein) that is dimensioned and configured to bind specifically with a small-molecule binding partner, such as a metal-containing ligand or other molecule.

ATF=artificial transcription factor. BSA=bovine serum albumin. DCC=N,N-dicyclohexylcarbodiimide. DMAP=dimethylaminopyridine. DMAPA dimethylaminopropylamine. DME=1,2-dimethoxyethane. DMF=N,N-dimethylformamide. DMSO=dimethylsulfoxide. DIEA=N,N-diisopropylethylamine. DTT=dithiothreitol. EMSA=electrophoretic mobility shift assay. ESI=electrospray ionization mass spectrometry. Fmoc=9-fluorenylmethyl chloroformate. HCCA=4-hydroxy-cyano-cinnamic acid. HEPES=N-[2-hydroxyethyl]piperazine-N′-[2-ethanesulfonic acid]. HOBt=1-hydroxybenzotriazole. HBTU=1-hydroxybenzotriazolyl-tetramethyl-uronium hexafluorophospate.

Nucleic acid=DNA, RNA, and modified forms thereof, including (without limitation), single, duplex, and triplex DNAs, homo-nucleic acids, hetero-nucleic acids, cross-overs, holliday junctions, bulges, bubbles, mismatches, hairpins, damaged nucleic acids, and nucleic acids incorporating non-standard base pairs.

MALDI-TOF=matrix-assisted laser-desorption ionization time-of-flight mass spectrometry. TFA=trifluoroacetic acid. TRIS=tris(hydroxymethyl)aminomethane. PA=polyamide.

PAM resin=Tert-butoxycarbonylaminoacyl-4-(oxymethyl)phenyl-acetamidomethyl-resin. It is commercially available and cleaved in high yield by aminolysis with primary amines. See Mitchell et al. (1978) J. Org. Chem. 43:2845.

PNA=peptide nucleic acid.

Proximate=When used in reference to the point where the anchor moiety is bonded to the target nucleic acid as compared to the anchor moiety's distance from the binding site, “proximate” means that the anchor moiety is disposed at a point sufficiently distant from the binding site so as not to alter the sequence or the conformation of the binding site. As a general rule, “proximate” denotes that the anchor moiety binds at a distance at least 2 nucleotides distant from either end of the binding site, and less than 500 nucleotides distant from either end of the binding site.

Regulatory factor=Any molecule, proteinaceous or otherwise, that regulates a biochemical reaction involving nucleic acids. Explicitly included within the term “regulatory factor” is any nucleic acid-binding ligand, including any molecule that plays a role in the activation or suppression of the transcription of a gene. Thus, the phrase “regulatory factor” explicitly includes (without limitation) transcription activators, transcription suppressors, transcription enhancers, transcription silencers, transcription co-factors, replication factors, recombination factors, stability factors, repair factors, splicing factors, localization factors, translation factors, and the like.

RFM=regulatory factor modulator.

TFM=transcription factor modulator.

Unless otherwise noted, the various techniques of molecular biology noted herein are well-known to those of skill in the art. Detailed protocols and guidance can be found in any of several well-known reference works, including: Sambrook, et al., “Molecular Cloning: A Laboratory Manual,” Cold Spring Harbor Laboratory Press (1989); Goeddel, ed., “Gene Expression Technology, Methods in Enzymology,” 185, Academic Press, San Diego, Calif. (1991); “Guide to Protein Purification” in Deutshcer, ed., “Methods in Enzymology,” Academic Press, San Diego, Calif. (1989); Innis, et al., “PCR Protocols: A Guide to Methods and Applications,” Academic Press, San Diego, Calif. (1990); Freshney, “Culture of Animal Cells: A Manual of Basic Technique, 2nd Ed.,” Alan Liss, Inc. New York, N.Y. (1987); Murray, ed., “Gene Transfer and Expression Protocols,” pp. 109-128, The Human Press Inc., Clifton, N.J.; Lewin, “Genes VI,” Oxford University Press, New York (1987); and “Current Protocols in Molecular Biology,” John Wiley & Sons, Inc, New York, N.Y. (1994-2004).

### DETAILED DESCRIPTION OF THE INVENTION

The principal utility of the present invention is a method to examine the molecular basis of nucleic acid binding properties (cooperative and otherwise) displayed by regulatory factors (for example, Hox proteins and their partners). Using the method, various test compounds can be be used to perturb, mimic, or otherwise modulate transcriptional networks that are dictated regulatory factors. Thus, the present method is useful to help elucidate the nature of nucleic acid binding and the role of opposing regulatory functions of regulatory factors (that is, the ability of a factor to enhance or initial transcription under one set of conditions, and to silence or suppress transcription under another set of conditions). The invention also contributes toward improving the precision with which ATFs can be designed, evaluated, and utilized to trigger specific transcriptional networks in vivo or in vitro.

For example, the present invention can be used to model molecular interfaces to create chemical mimics of, for example, Hox proteins. A wealth of phenotypes have been associated with various mutations of the Ultrabithorax gene (Ubx) in Drosophila. Even subtle effects caused by a decrease in Ubx dosage can be readily identified. In general, Hox proteins, especially from Drosophila, offer several advantages as a model system to demonstrate the utility and functionality of the present invention. First there exists a large body of genetic, biochemical, structural and phenotypic information on the roles of various homeodomain-bearing proteins in Drosophila. Additionally, the Drosophila genome is sequenced and a bank of genetic lesions in every annotated gene is being systematically compiled. Microarray-based, genome-wide gene expression analysis of the Drosophila transcriptome is possible.

Homeodomain is a 60-residue helix-loop-helix motif that binds DNA. Although they can bind as monomers to regulate the expression of certain genes, Hox proteins also bind to the Pbc family of atypical homeodomain proteins and only as heterodimers do Hox proteins display high sequence specificity for their DNA target sites. Often additional DNA binding partners like Homothorax (Hth) have also been shown to bind promoters as a Hox-Pbc-Hth complex. This would increase the sequence that is recognized and thus limit the number of genes that are regulated by the ternary complex. In short, the evidence compiled to date strongly indicates that the exquisite sequence specificity displayed by regulatory factors such as transcription factors, is a tightly controlled, coordinated process.

The structures of Ubx in complex with its Pbc partner—Extradenticle (Exd) and a human paralog HoxBl in complex with Pbxl are reported in the literature. Remarkably, the crystal structures of the DNA-bound Drosophila Ubx-Exd and the human HoxBl-Pbxl complex are highly similar and both structures show a conserved peptide (YPWMIFDWM) (SEQ. ID. NO: 9), contributed by Ubx/HoxBl. This peptide-Exd interface is thought to contribute to the cooperative binding of Ubx-Exd and HoxBl-Pbxl to specific DNA sites. However, it has been argued that allosteric modulations of DNA geometry and additional protein contacts contribute significantly to cooperative binding as well. Thus, the present invention can be used to mimic (and thus to model and to evaluate) the DNA binding properties of Ubx or HoxBl (an illustrative and non-limiting example) and its peptide interface with Exd or Pbxl. This is accomplished using a suitable test compound, such as the conserved docking peptide, bonded to a flexible chemical linker, that is then bonded to a polyamide designed to bind proximate to the relevant DNA sequence.

The minor groove next to the Exd YPWM (SEQ. ID. NO: 10) binding site is sufficiently wide (12.6 Å, as compared to 13 Å in a polyamide crystal structure) to accommodate a hairpin polyamide. Moreover, the C-terminal methionine residue of the YPWM peptide is pointing toward this minor groove, and turning away from the proximal backbone phosphate. Attachment of the YPWM motif to the polyamide residue located above the nearest nucleotide base yields a very short linker, thus achieving maximum cooperation. Using the present invention, the energetic contribution of docking peptide-Exd interaction to the cooperation displayed by these two Hox proteins and Exd in binding to their respective sites can be determined.

Hairpin polyamides 1 and 2 (see Examples) have been designed to match the respective binding sites. To measure the energetic benefits afforded by interactions between the polyamide-Y(P/K)WM (SEQ. ID. NO: 11) conjugates and Exd, quantitative footprint titration assays, isothermal calorimetric analysis, and fluorescence anisotropy binding studies can be performed. The present invention can also be used to determine the extent to which the DNA binding sites for polyamide-YPWM and the Exd can be separated, while still retaining the cooperative binding to DNA. The invention can also be used to determine the affinity of the polyamide conjugates for Exd in the presence of DNA, as well as to provide biophysical insight into the role of linker length in defining the cooperative binding to adjacent sites on the DNA target. Moreover, using the present invention, the energetic contribution of peptide-Exd interaction can be unambiguously delineated from all other possible direct or allosteric events that contribute to cooperative DNA binding by the two Hox/Exd complexes.

The invention can also evaluate in vivo function and examine the role of opposing regulatory functions. For example, the compositions of matter described herein can be fed to first instar Drosohila larvae and their effect on various developmental pathways that are influenced both by, for example Ubx and Lab, can be determined. Another mode of delivery would be to microinject polyamide conjugates into embryos. In either case, the polyamides would be coupled to carrier peptides to facilitate their mobility into cells.

The present method can be used to examine and characterize chemical mimics of human proteins. In short, the exemplary strategy described herein to target Ubx-Exd interactions in Drosophila can be extended to design ATFs that substitute for the human homeodomain paralogs. The most direct approach, due to the near identical binding of the tetrapeptides of the HoxBl to Pbx-1, would be to substitute HoxBl with polyamides conjugated to FDWM peptide. Unlike Drosophila, where cultured cells are thought not to reproduce features of Ubx regulation faithfully, cell culture studies can be performed with a variety of cell lines immortalized by mutated or overexpressed Hox proteins. By coupling carrier peptides to compositions of matter as described herein that mimic HoxBl, and then following their regulatory effects on the transcriptome (by microarray analysis) the key nodes in transcriptional networks that are regulated by this Hox-Pbx-1 complex can be identified. It should be noted that this information is particularly valuable because malfunctions of other human Hox paralogs have been implicated in leukemic transformations.

Going beyond homeodomains, the present invention can also be used to target different classes of DNA binding modules. Cooperative binding at composite DNA binding sites is a common property of eukaryotic regulators. For example, the interface between NFAT, a calcium-responsive factor in activated T-cells, and activator protein 1 (AP-1) was shown to play a role in cooperative binding by both partners at the interleukin-2 promoter. In another example, in addition to favorable cooperative binding between Ets-1 and Pax5, interfacial molecular interactions alter the DNA sequence specificity of Ets-1. These examples emphasize the underlying principle of weak molecular interfaces, stabilized on DNA, that strongly influence the choice of promoters targeted by transcription factors. Thus, the present invention can be used to screen peptide and small molecule libraries to seek molecules that will interact with members of different classes of regulatory factors in general and transcription factors in particular. The peptides or small molecules that show specific binding to DNA binding domains (ideally derived from developmental-stage or cell-type specific transcriptional factors) would then be characterized further via functional assays in cell culture and in model organisms.

In summary, the present invention will provide powerful tools that can be used to study intractable mechanistic features of transcriptional regulation, they can serve as tools to dissect genome-wide transcriptional networks, and they can be used as guides to trigger desired transcriptional cascades that control cell fate.

The Defined Binding Site in the Nucleic Acid Target:

The binding site to be studied in the nucleic acid target can be any regulatory factor binding site, without limitation. Exemplary binding sites that can be defined within the nucleic acid target include, without limitation, promoter binding sites, transcription factor binding sites, enhancer binding sites, silencer binding sites, suppressor binding sites, and the like. A promoter is a regulatory sequence of DNA that is involved in the binding of RNA polymerase to initiated transcription of a gene. An enhancer is regulatory sequence of DNA that can increase the utilization of promoters, and can function in either orientation (5′-3′ or 3′-5′) and in any location (upstream or downstream) relative to the promoter. A silencer sequence or suppressor sequence generally has a negative regulatory effect on expression of the gene.

The Anchor Moiety:

The anchor moiety can be any moiety dimensioned and configured to yield a robust bond of the anchor to the nucleic acid target under physiological conditions. The anchor may be covalently linked to the nucleic acid target, or the anchor may be conjugated to the nucleic acid target.

If the anchor moiety is covalently linked to the nucleic acid target, the covalent bond linking the anchor moiety to the nucleic acid target can be formed using any of several well known chemistries now known in the art. For example, the anchor moiety can be linked to the nucleic acid backbone via a phosphothioether or phosphothioester bond between the anchor moiety and the phosphates present in the nucleic acid.

Covalent bonds to nucleic acids may also be formed using any of a number of alkylating agents, for example, nitrogen mustards (which alkylate nucleic acids mainly through the 7-position nitrogen atom of guanine although other moieties can also be alkylated), nitrosoureas, and the like.

Thiol-independent nucleic acid alkylation can be accomplished using the method of Gates et al. (2001) J. Amer. Chem. Soc. 123(9):2060-2061, incorporated herein by reference. In Gates' approach, the antitumor/antibiotic agent leinamycin is used as a means to alkylate a DNA target independent of a thiol-mediated reaction. Briefly, an isolated DNA target to be alkylated is reacted with leinamycin, in the absence of thiol. The alkylation pattern resulting from the thiol-free reaction is identical to the analogous reaction in the presence of thiol, but occurs more slowly and yields roughly 30% of the alkylated product as compared to the thiol-mediated reaction. Without being limited to a particular mechanistic pathway, the thiol-independent mechanism of alkylation is believed to proceed by attack of water (or hydroxide) on the C3′-carbonyl of the leinamycin to yield a sulfenic acid intermediate. An intramolecular rearrangement involving the attack of a neighboring carboxylate group on the sulfenic acid group results in an oxathiolanone intermediate. This results in alkylation of the DNA target via an episulfonium ion. The reaction efficiently alklyates duplex DNA and the N7 position of guanine residues. See also Asai et al. (1996) J. Amer. Chem. Soc., 118:6802-6803.

Polycyclic aromatic hydrocarbons are also known to form covalent bonds with nucleic acids.

Well-known pharmacological agents can be utilized for their ability to bind to nucleic acids. For example, mitomycin C, cisplatin, and anthramycin all form covalent bonds with DNA, and can act as the anchor moiety in the present invention. Mitomycin C is a well-characterized antitumor antibiotic that forms a covalent interaction with DNA after reductive activation. The activated antibiotic forms a cross-linking structure between guanine bases on adjacent strands of DNA thereby inhibiting single strand formation. Anthramycin is an antitumor antibiotic which binds covalently to N-2 of guanine located in the minor groove of DNA. Anthramycin has a preference of purine-G-purine sequences, with bonding occurring at the middle G. Cisplatin is a transition metal complex cis-diamine-dichloroplatinum and is clinically used as anti-cancer drug. The effect of the drug is due to the ability to platinate the N-7 of guanine on the major groove site of DNA double helix. This same effect can be used to serve as an anchor moiety in the present invention.

Intercalators are a class of molecules which are potent antibiotic and antitumor drugs. Lerman first described intercalation as the insertion of a flat, aromatic chromophore between adjacent base pairs of the double helix. See Lerman (1961) J. Mol. Biol. 3:18-30. The rise between base pairs in B-form DNA is usually 3.4 Å/base pair. The insertion of the intercalator separates the adjacent base pairs by another 3.4 Å and extends the length of the helix an equivalent amount per bound intercalator. The base pairs neighboring the intercalation site are also unwound 10-26° with respect to one another. Generally, it is these structural distortions introduced by intercalation which are considered to be the basis for their therapeutic activity. In most cases, the DNA helix returns to its B-form structure within a few base pairs of the intercalation site. Because they bind strongly with DNA, intercalators can be used as anchor moieties in the present invention.

The anchor moiety can also be a polyamide as described in U.S. Pat. No. 6,506,906, issued Jan. 14, 2003, and published PCT patent application WO 02/34295, published May 2, 2002, both of which are incorporated herein. Polyamides (PAs) are the preferred anchor moiety for use in the present invention due to their sequence specificity and strong DNA binding affinity.

The preferred PA comprises the following subunits: 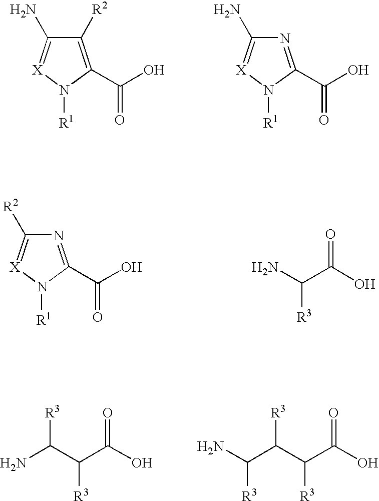
wherein R¹ is C₁₋₁₀₀ alkyl, C₁₋₁₀₀ alkylamine, C₁₋₁₀₀ alkyldiamine, C₁₋₁₀₀ alkylcarboxylate, C₁₋₁₀₀ alkenyl, C₁₋₁₀₀ alkynyl, or C₁₋₁₀₀ L (and in all cases the C₁₋₃₀ homologs being preferred);
wherein L is selected from the group consisting of arylboronic acid, biotin, polyhistidine comprising from 2 to 8 amino acids, a hapten to which an antibody binds, a solid-phase support, oligodeoxynucleotide, N-ethylnitrosourea, fluorescein, bromoacetamide, iodoacetamide, DL-lipoic acid, acridine, captothesin, pyrene, mitomycin, texas red, anthracene, anthrinilic acid, avidin, DAPI, isosulfan blue, malachite green, psoralen, ethyl red, 4-(psoralen-8-yloxy)-butyrate, tartaric acid, and (±)-tocopheral;
wherein m is an integer value ranging from 0 to 12;
R² is H, NH₂, SH, Cl, Br, F, N-acetyl, or N-formyl;
R3 is H, NH₂, OH, SH, Br, Cl, F, OMe, CH₂OH, CH₂SH, or CH₂NH₂; and
X is N, CH, COH, CCH₃, CNH₃, CCl, or CF.

Baird et al. (1996) J. Am. Chem. Soc., 118:6141-6146m and PCT/US97/003332 describe methods for synthesizing polyamides suitable for use in the present invention. Polyamides may be synthesized by solid-phase methods using compounds such as Boc-protected 3-methoxypyrrole, imidazole, and pyrrole aromatic amino acids, which are cleaved from the support by aminolysis, deprotected with sodium thiophenoxide, and purified by reverse-phase HPLC. The identity and purity of the polyamides may be verified using any number of analytical techniques available to one skilled in the art such as ¹H-NMR, analytical HPLC, and/or MALDI-TOF MS.

In addition, the above polyamide subunits can be synthesized in small scale by methods known in the art. See Grehn & Ragnarsson (1981) J. Org. Chem. 46: 3492; and Grehn et al. (1990) Acta. Chim. Scand. 44:67;

The polyamide polymer can be a homopolymer of Py and Im subunits or a copolymer with strategically placed aliphatic amino acid monomers such as α-amino acids (including but not limited to the naturally occurring amino acids and preferably being glycine), and amino acids of the formula —NH—(CH)ₙ—CO—, where “n” is an integer from 1-12 (preferably “n” being 1 as in -alanine or 2 as in -aminobutyric acid).

The carboxy terminus of the polyamide may comprise, for example, NH(CH₂)₀₋₆, NR¹R² or NH(CH₂)_{b}CONH(CH₂)₀₋₆NR¹R², NHR¹ or NH(CH₂)_{b}CONHR¹, where b is an integer from 1-6 and R¹ and R² are independently chosen from C₁₋₆ alkyl, C₁₋₆ alkylamine, C₁₋₆ alkyldiamine, C₁₋₆ alkylcarboxylate, C₁₋₆ alkenyl, C₁₋₆ alkynyl, or a C₁₋₆L (where L is as described previously).

Solid-phase synthesis involves the step-wise assembly of a molecule while one end is covalently anchored to an insoluble matrix at all stages of the synthesis. See, for example, Merrifield (1963) Am. Chem. Soc. 85:2149-2154; and Merrifield (1986) Science 232:341-347. In the 40-odd years since solid-phase synthesis was first invented, general protocols have been developed for manual and machine-assisted Boc-chemistry solid-phase synthesis of polypeptides and polyamides of all sorts, including pyrrole-imidazole polyamides. See, for example, Baird & Dervan (1996) J. Am. Chem. Soc. 118:6141, incorporated herein.

Polyamides containing more than 4 residues are preferably prepared by solid phase methodology. For solid phase synthesis, the polyamide is attached to an insoluble matrix by a linkage which is cleaved by a single step process which introduces a positive charge into the polyamide. The addition of an aliphatic amino acid at the C-terminus of the polyamides allows the use of Boc-alanine-Pam-Resin (which is commercially available in appropriate substitution levels [0.2 mmol/gram]). Aminolysis of the resin-ester linkage provides a simple and efficient method for cleaving the polyamide from the support. See Mitchell et al. (1978) J. Org. Chem. 43:2845.

Suitable synthetic methods are also described in Schnolzer et al. (1992) Int. J. Peptide. Protein. Res. 40:180; and Milton et al. (1992) Science 256:1445. As a general rule, coupling cycles are rapid (72 min per residue for manual synthesis or 180 min per residue for machine-assisted synthesis), and require no special precautions beyond those used for ordinary solid-phase peptide synthesis. The manual solid-phase protocol for synthesis of polyamides has been optimized for automatic synthesis on an ABI 430A peptide synthesizer. Step-wise cleavage of a sample of resin and analysis by HPLC indicates that high step-wise yields (>99%) are routinely achieved.

The Linker Moiety:

The linker moiety is preferably a linear or branched, bifunctional aliphatic linker, or a cyclical, heterocyclical, aromatic, or heteroaromatic bifunctional linker, having a length (along its major axis) of no more than about 40 Å. For aliphatic linkers, the backbone of the linker will generally have from 1 to about 50 atoms.

Preferred linkers including alkyl, alkenyl, alkynyl linkers, or alkylamino, akenylamino, alkynylamino linkers, having from 1 to about 50 carbon atoms, and more preferably still a homo- or hetero-polypeptide having from 1 to 16 residues, with 1-10 residues being preferred (e.g, poly(glycine), poly(proline), etc.). A peptide of from 4 to 16 residues and incorporating the motif Xxx-Xxx-W-M (where Xxx is any α-, β-, or γ-amino acid, natural or artificial) is the preferred polypeptidic linker. From among this class of polypeptides, the preferred motifs are YPWM (SEQ. ID. NO: 10), YKWM (SEQ. ID. NO: 11), and FDWM (SEQ. ID. NO: 12).

Poly(alkylene)glycols, such as poly(ethylene)glycol (PEG) and poly(propylene)glycol can also be used as the linker moiety. See Example 7.

Of particular note with regard to the linker is that its length and entropy play a critical role in determining the solvent space that is ultimately accessible to the test compound. The longer and more flexible the linker, the more solvent space that can be accessed by the test compound bonded to the linker.

Multivalent interactions are frequently encountered in biological systems. Typically, the monovalent features of these molecular interactions are weak and utilize a small surface area between the interacting biomolecules. These features are reiterated, often in a modular fashion, and the resulting multivalent interaction greatly improves the association between the two biomolecules. While the multivalent association may or may not be cooperative, it does significantly improve association between interacting partners. This principle of multivalent binding has been utilized in the design of highly stable organometallic complexes (e.g., organo-metallic chelates). In drug design, small molecule fragments that individually bind weakly to a target protein have been identified by NMR and then linked to each other to generate bivalent ligands that associate more strongly with the target protein than either molecule separately. See Maly, Choong & Ellman (March 2000) PNAS 97(6):2419-2424. In a more recent approach small molecules that bind weakly to a particular surface of a target protein are tethered by a disulfide exchange to an engineered cysteine. Subsequently these small molecules/fragments are used to identify additional fragments that bind adjacent surfaces. The resulting composite molecule displays a greatly improved overall affinity for the target protein due to “multivalent” interactions. See Erlanson et al. (August 2000) PNAS 97(17):9367-9372. In these reported works, the linkers were designed to be as short as possible to minimize entropic costs, but at the same time were designed with sufficient (but limited) flexibility to permit multivalent associations.

As shown in the Examples, the compositions of matter described herein can greatly improve the affinity of a targeted DNA binding protein for its specific DNA recognition sequence. At the same time, the identical composition of matter is ineffective at sites where the DNA sequence does not match the preferred DNA binding site of the target protein. In contrast to strategies that target adjacent surfaces on a protein to create a bivalent ligand, in the present approach, both a DNA binding site, as well as a linked test compound, are used to generate a “bivalent” surface that enhances the association of the targeted regulatory factor with its cognate nucleic acid site. The length and flexibility of the linker moiety is one parameter of the compositions that can be altered to optimize any given interaction between a regulatory factor and its corresponding nucleic acid binding site.

In the initial research leading to the present invention, short hydrocarbon linkers were used to conjugate the anchor moiety to the test compound. Example 7, however, describes the effects of varying linker length on the ability of a composition of matter according to the present invention to recruit a DNA-binding protein efficiently.

As shown in Example 7, the anchor moiety is a sequence-specific hairpin polyamide that is composed of N-methylpyrrole (Py) and N-methylimidazole (Im) hetrocycles linked via amide bonds. The test compound, also referred to as the “hook,” is a conserved tetra-peptide (YPWM) (SEQ. ID. NO: 10) derived from the Hox-family of transcription factors. The Hox tetra-peptide interacts with Extradenticle (Exd)—a DNA-binding protein—and stabilizes the assembly of a ternary Hox-Exd-DNA complex. The crystal structure of the Hox-Exd-DNA complex was used to guide the design of a synthetic molecule that would present the YPWM peptide hook adjacent to the DNA binding site and stabilize the association of Exd with DNA. The polyamide anchor moiety of the composition was conjugated to the YPWM test compound using a propyl linker. This synthetic molecule efficiently mimics the ability of the natural Hox protein to stabilize Exd binding to DNA.

Example 7 explores the role of the linker in determining the effectiveness of these compounds to recruit Exd. Thus, the goals of Example 7 were: a) to determine how far a test compound can be positioned from the nucleic acid target without significant loss in effectiveness; b) determine the optimal length of the linker.

In Example 7, eight different linkers were used, ranging in length from about about 5 Å to about 32 Å. As noted in Example 7, at low temperatures a test compound attached to linker that is ˜32 Å long still effectively recruit the DNA binding protein to the adjacent binding site on the nucleic acid target. This permits access to a much larger surface of the regulatory factor in the selection of test compounds that might bind to unique surfaces of a desired regulatory factor. The results of Example 7 strongly suggest that prior knowledge of the structure of the regulatory factor of interest is not necessary to identify test compounds that may bind specifically to rigid or flexible surfaces of, for example, DNA binding proteins. Thus, as a general proposition, a longer linker helps overcome a key stumbling block in structure-based design, namely being limited to examining surfaces that are rigid and have been precisely mapped structurally.

Not surprisingly, increasing linker length inflicts an energetic penalty on the ability of the test compound to recruit the regulatory factor to an adjacent nucleic acid binding site. However, this penalty can be tuned such that a bifunctional molecule capable of functioning at lower temperatures is rendered incapable of binding under physiological temperatures. Thus, rather than minimizing linker entropy when creating multivalent ligands (as in the prior art), by destabilizing the linker (via increased entropy) the linker creates a conditional “chemical switch.” Temperature sensitivity thus permits rapid spatio-temporal control of the activity of the test compound bonded to the linker. The utility of this approach is that the flexible linker will behave differently at different temperatures, due to the increased entropy inherent in a longer linker. This characteristic of entropically destabilized linkers is designated herein as “conditional behavior.”

The linker can also be designed as an aptamer that can self-assemble around a second small molecule of interest. By designing the linker to mate specifically with another small molecule, the linker can be made to function as a ligand-gated chemical switch. In other words, the linker behaves in a first manner in the absence of its binding partner, and in a second manner (different from the first) in the presence of its binding partner.

A host of aptamers are known in the art and are suitable for use in the present invention. For example, an anti-thrombin aptamer has been generated against thrombin. This aptamer has been extensively studied in a variety of animal models of anti-coagulation. See, for example, Boch et al. (1992) Nature 355:564. In several of these studies, the aptamer was shown to be as effective as heparin at systemic anticoagulation. Such an aptamer can be utilized as the linking moiety in the present invention.

Aptamers are also known that bind selectively with such biological entities as platelet-derived growth factor B (PDGF-B) (Floege et al. (1999) Am. J. Pathol. 154:169); transforming growth factor βs (TGFβ2) (Cordeiro et al. (2000) Eye 14:536-47); L-selectin (Hicke et al. (1996) J. Clin. Invest. 98:2688); neutrophil elastase (Bless et al. (1997) Curr. Biol. 7:877); complement C5 (Biesecker et al. (1999) Immunopharm. 42:219); and keratinocyte growth factor (KGF) (Pagratis et al. (1997) Nat. Biotech. 15:68.) Aptamers can also be purchased commercially from a number of suppliers, including Archemix Corp., Cambridge, Mass.

All are suitable for use in the present invention.

The Test Compound:

The test compound can be any moiety, without limitation, that is desired to be tested for its ability to modulate binding of regulatory factors to a nucleic acid target.

Reaction Conditions:

As used herein, the phrase “under transcription conditions” explicitly denotes conducting the given experiment under physiological conditions where transcription would take place if all of the required ingredients necessary for transcription were present. In short, the term does not require that transcription take place (although it does explicit encompass those conditions where transcription does actually occur), or even that all of the required entities for transcription be present in the reaction mixture. Generally, “transcription conditions” denotes a reaction environment of 37° C., and having pH, ionic strength, and reduction conditions that are within physiological ranges (or under gently reducing conditions). Suitable reaction buffer mixes are available from a host of commercial suppliers, including Promega Corporation (Madison, Wis.) and Ambion Inc. (Austin, Tex.).

An exemplary reaction mixture (non-limiting) that contains all of the ingredients required for in vitro transcription is as follows:
1 μL transcription buffer (Ambion)
1 μL NTPs (4 mM ATP and CTP, 1 mM GTP and UTP)
2 μL 10 mM GpppG cap (Pharmacia)
2 μl ³²P-UTP, 800 Ci/mMol (NEN)
0.2 μL Rnasin (Promega)
1 μL 0.1 M MDTT
1.8 μL H₂O
0.5 μL isolated nucleic acid target (1 μg/μL)
0.5 μL polymerase (SP6/T7/T3)
   The ingredients are combined and the reaction mixture is incubated for 1 hour at 37° C. See also the Examples for additional exemplary protocols.
   Measuring the Results of the Reaction:

Determining whether the test compound alters binding of the natural transcription factor to the modified nucleic acid can be done by any number of means, including electorphoretic gel shift, fluorescence polarization spectroscopy, x-ray crystallography, Biacore-type affinity spectroscopy, nuclear magnetic resonance spectroscopy, circular dichroic spectroscopy, quantitative DNase 1 footprinting assays, etc. These techniques are well-known to those skilled in the art and will not be described in any detail herein.

For example, affinity cleaving titration experiments (25 mM Tris-Acetate, 20 mM NaCl, 100 mM bp calf thymus DNA, pH 7, 22° C., 10 mM DTT, 10 mM Fe(II)) using polyamides modified with EDTA•Fe(II) at the C-terminus can be used to determine oriented binding. MPE•Fe(II) footprinting experiments can be used to determine binding site size. See Hertzberg & Dervan (1982) J. Am. Chem. Soc., 104:313 (1982); Van Dyke & Dervan (1983) Biochemistry 22:2373; Van Dyke & Dervan (1983) Nucleic Acids Res. 11:5555; and Hertzberg & Dervan (1984), Biochemistry 23:3934. Typical reaction conditions are: 25 mM Tris-acetate, 10 mM NaCl, 100 μM calf thymus DNA, 5 mM DTT, pH 7.0, and 22° C.

Quantitative DNaseI footprinting can be used to determine the equilibrium association constants for binding to match and mismatch sites. Footprinting experiments are generally performed on 3′- and/or 5′-³²P end restriction fragments derived from plasmids. 3′-shifted cleavage patterns are consistent with location of the polyamide in the minor groove. Typical reaction conditions are: 10 mM Tris-HCl, 10 mM KCl, 10 mM MgCl₂, 5 mM CaCl₂, pH 7.0, and 22° C. See Brenowitz et al. (1986). Methods Enzymol. 130:132-181; Fox & Waring (1984) Nucleic Acids Res. 12:9271-9285; and Brenowitz et al. (1986) Proc. Natl. Acad. Sci. U.S.A. 83: 8462-8466.

### EXAMPLES

The following Examples are included solely to provide a more complete understanding of the invention disclosed and claimed herein. The Examples do not limit the scope of the invention in any fashion.

Materials: Boc-Ala-PAM resin (0.59 mmol/g), anhydrous HOBt and HBTU were purchased from Peptides International (Louisville, Ky.). “SASRIN′-brand resin and all Fmoc/tBu protected-amino acids were from Bachem (Bubendorf, Switzerland), TFA was from Halocarbon (River Edge, N.J.), and DMSO was from Fisher Scientific (Hampton, N.H.). All other solvents and reagents were anhydrous and/or ACS-grade, purchased from VWR (West Chester, Pa.) or Aldrich (Milwaukee, Wis.), and used as received. Water was purified using a Millipore MilliQ water purification system (18 MΩ). Biochemical experiments were performed using RNase-free water (Invitrogen, Carlsbad, Calif.). DNase I and calf thymus DNA were purchased from Amersham (Piscataway, N.J.). All other enzymes and materials for molecular biology were from Roche (Nutley, N.J.). All buffers were 0.2 μm filtered before storage. Oligonucleotide oligomers were from Integrated DNA Technologies Inc. (Coralville, Ia.).

Methods: UV spectra were recorded on a HPS4S2A diode array spectrophotometer. All polyamide compound concentrations were determined by UV spectroscopy (H₂O) employing ε=69500 L mol⁻¹ cm⁻¹ at λₘₐₓ near 312 nm. ESI and MALDI-TOF mass spectra were recorded on a Finnigan LC-Q (2 μM in 50% acetonitrile, 5 μL/min) or a Perseptive Biosystems Voyager instrument (5 pmol samples in 4-HCCA matrix). Analytical UPLC was performed on a Beckman Gold HPLC System fitted with a diode array detector and a Varian-RP18 microsorb column (250×4.6 mm) at 1 mL/min, 0-100% CH₃CN in 0.1% TFA (v/v) in 30 min. Preparative HPLC was performed on a Beckman Gold HPLC System fitted with a diode array detector and a Waters DeltaPak-RP18 column (25×100 mm) equipped with a guard, at 8 mL/min (0-50% CH₃CN in 0.1% TFA in 50 mm, Method #1), or a DeltaPak-RP18 column (25×100 mm) equipped with a guard attached to a Varian Dynamax-RP18 column (21.4×250 mm), at 16 mL/min (0-40% CH₃CN in 0.1% TFA in 70 min, Method #2).

### Example 1

### Synthesis of Polyamide Anchor Moieties

Polyamide 1 was synthesized by manual solid phase synthesis following established procedures. Cleavage from PAM resin was accomplished by aminolysis with neat DMAPA (37° C., 12 h). The volatiles were removed in vacuo, the residue taken up in 10% AcOH and purified by prep. HPLC (Method #2). HPLC 14.6 mm. MS (ESI) [M+H]⁺ calcd for C₅₉H₇₆H₂₃O₁₀ 1266.6, found 1266.4.

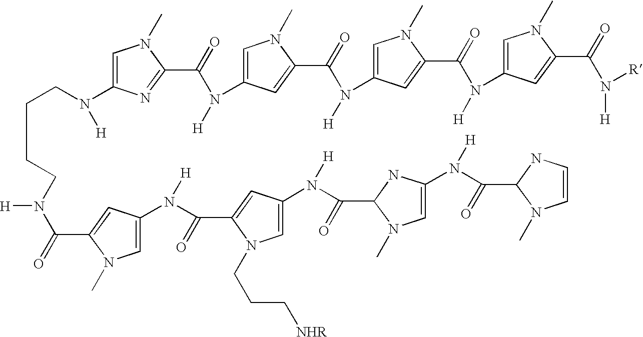

1 (R=H)

(SEQ. ID. NO: 2)

2 (R=Ac-Phe-Tyr-Pro-Trp-Met-Lys-Gly-)

(SEQ. ID. NO: 3)

3 (R=Ac-Phe-Tyr-Pro-Ala-Ala-Lys-Gly-)

### Example 2

### Synthesis of Modular Glycine Linker and Polypeptide Test Compound

tBu-protected peptide acids were synthesized by manual solid phase synthesis on SASRIN™ resin. In brief, 125 mg of SASRIN™ resin (1.08 mmol eq/g) were placed in a presiliconized peptide synthesis vessel, preswollen in CH₂Cl₂ (10 min), and combined with a premixed (30 min) and filtered solution of Fmoc-Gly-OH (150 mg, 0.5 mmol, 4 eq) in DMF (125 μL) and DCC (500 μL, 1.0 M in CH₂Cl₂, 0.5 mmol, 4 eq). DMAP (6 mg, 0.05 mmol, 0.1 eq) was added, and the mixture was shaken for 12 h. After draining and washing (CH₂Cl₂, DMF, CH₂Cl₂), the loaded resin was capped by treatment with benzoyl chloride/pyridine/CH₂Cl₂ 1:1:3 (1.25 mL) for 30 min. Fmoc deprotection was in general achieved by treatment with 25% piperidine in DME (3×: 2 sec, 30 sec., and 15 min), but the second residue was deprotected with 50% piperidine in DMF (3×: 2 sec, 30 sec, and 5 min). Amino acid coupling was performed for 1.5 h at room temperature using a solution of 0.3 mmol Fmoc/tBu protected amino acid in DMF (0.7 mL) preactivated with 0.3 mmol HOBt, 0.27 mmol HBTU and 50 μL of DIEA for 5 min. After 15 min of coupling time, more DIEA (20 μL) was added to the mixture. After successive build-up of the peptide chain, the terminal Fmoc group was removed and the resin-bound peptide treated with a mixture of Ac₂O/pyridine/DMF 2:3.10 (1 mL) for 30 min followed by thorough washing (DMF, iPrOH, DMF, CH₂Cl₂). The peptide was cleaved from the resin in four cycles, where the resin was treated with TFA/ethanedithiol/Et₃SiH/CH₂Cl₂ (1:5:5:89) (1.5 mL) for 15 min. After each cycle, the resin was drained, and the obtained solution was immediately cooled to 0° C. and neutralized with pyridine (20 μL). All cleavage solutions were combined and partitioned between EtOAc (70 mL) and 0.1 M KHSO₄ (30 mL). The organic layer was washed with brine (2×20 mL), dried with Na₂SO₄, and the volatiles were evaporated. Purification of the residue by flash column chromatography (20 g of silica) yielded the pure peptide acids. Peptide 4: Yield 148.5 mg (121 μmol, 88%); TLC (CH₂Cl₂/MeOH/HCOOH 100:5:1) R_{f}0.16; HPLC 23.4 min MS (ESI, neg.) [M−H]⁻ calcd for C₆₃H₈₆N₉O₁₄S 1224.6, found 1224.5. Peptide 5: Yield 54.2 mg (57 μmol, 42%); TLC (CH₂Cl₂/MeOH/HCOOH 100:10:1) R_{f} 0.19; HPLC 18.0 min; MS (ESI, neg.) [M−H]⁻ calcd for C₄₈H₆₉N₈O₁₂ 949.5, found 949.5.

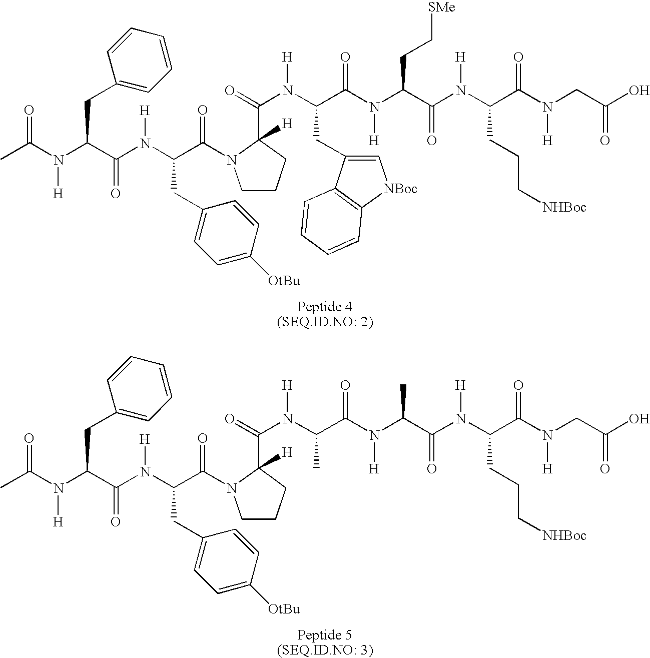

### Example 3

### Binding Anchor Moiety to Linker Moiety and Test Compound

A solution of 10 μmol (4 eq.) of the respective peptide acid in CH₂Cl₂/DMF 10:1 (2.5 mL) was treated at room temperature with 0.1 M HBTU in DMF (110 μL, 11 μmol) and 1.0 M DIEA in DMF (12 μL, 12 μmol) for 5 min, before approx. 2.5 μmol of polyamide 1 TFA salt in DMF (2.5 mL) were added, followed by 12 μL of 1.0 M DIEA in DMF. After the conversion was complete (2 h, HPLC control), the volatiles were removed in vacuo, and the residue was dissolved in TFA/CH₂Cl₂/ethanedithiol/Et₃SiH (80:10:5:5) (1 mL). After 20 min, the crude peptides were precipitated with cold Et₂O (10 mL, 0° C.) and isolated by centrifugation and discarding of the supernatant. The colorless powder was resuspended twice in Et₂O (5 mL, 0° C.), isolated by centrifugation, and then taken up in 0.2 M AcOH. After standing for 4 h, this solution was purified by prep. HPLC (Method #1) to yield the conjugates in >97.5% HPLC purity (312 nm). Conjugate 2: Yield 3.7 mg (1.66 μmol, 62%) from 4 and 2.67 μmol 1; HPLC 17.0 min; MS (MALDI-TOF) [M+H]⁺ calcd for C₁₀₈H₁₃₇N₃₂O₁₉S 2218.1, found 2218.0. Conjugate 3: Yield 1.4 mg (0.72 μmol, 26%) from 5 and 2.8 μmol 1; HPLC 15.5 min; MS (MALDI-TOF) [M+H]⁺ calcd for C₉₈H₁₂₈N₃₁O_{·S} 2043.0, found 2042.9.

### Example 4

### Determining Dissociation Constants for Binding to DNA

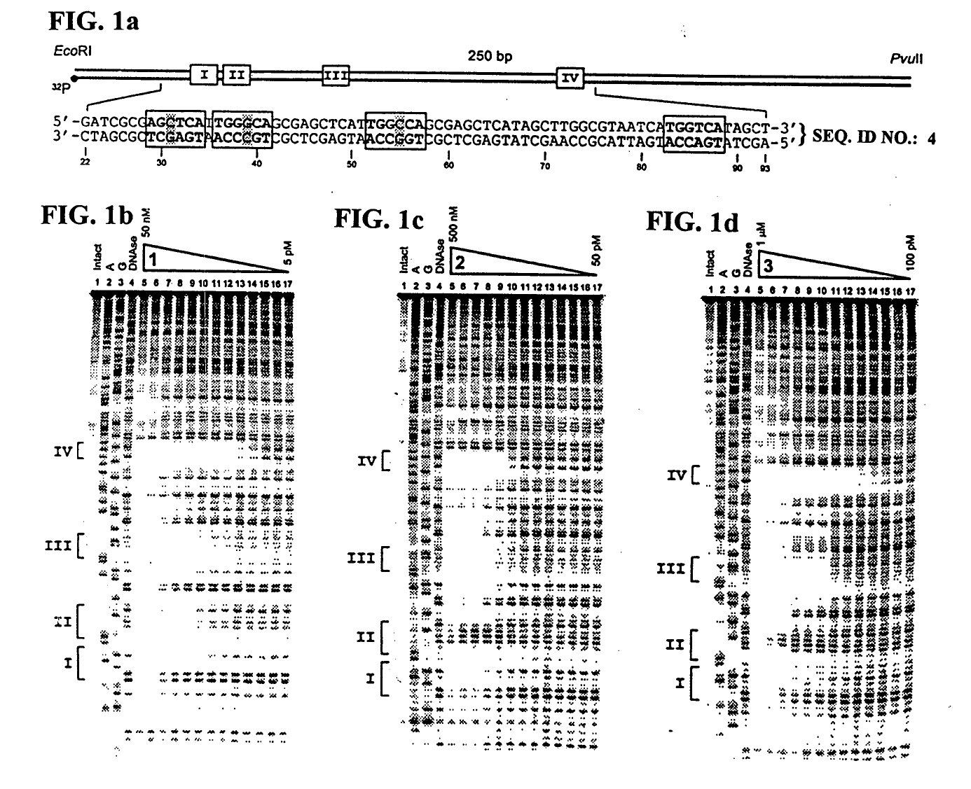

DNase I Footprinting: Dissociation constants for the DNA binding of compounds 1, 2 and 3 were obtained following published protocols. All reactions were carried out in 400 μL total volume employing 20 kcpm of a 3′-radiolabelled 250-bp restriction fragment from the plasmid pDEH9 (FIG. 1a) (SEQ. ID. NO: 4). No carrier DNA was used in the equilibration, and the solutions were allowed to equilibrate for 12 h at 22° C. in TKMC buffer (10 mM TRIS, 10 mM KCl, 5 mM MgCl₂, 5 mM CaCl₂ pH 7.0) prior to the DNase I digestion. Reaction products (8 kcpm) were resolved on denaturing 8% polyacrylamide sequencing gels run at 55 W.

FIG. 1a: The overall composition and insert sequence of the EcoRl/PvuII restriction fragment from the plasmid pDEH9. Polyamide binding sites are highlighted with boxes, mismatched base pairs are shaded in gray. The site of the 3′-³²P-labeling is indicated (lower strand).

FIGS. 1b, 1c, and 1d are quantitative DNAse I footprint titration experiments for compounds 1, 2 and 3 on the 3′⁻³² P-labeled 250-bp EcoRI/PvuII restriction fragment from the plasmid pDEH9. Lane 1: Intact DNA. Lane 2: A-reaction. Lane 3: G-reaction. Lane 4: DNAse 1 standard.

FIG. 1b: Compound 1: Lanes 6-17: 50 nM, 20 nM, 10 nM, 5 nM, 2 rM, 1 nM, 500 pM, 200 pM, 100 pM, 50 pM, 20 pM, 10 pM, 5 pM, respectively.

FIG. 1c: Compound 2: Lanes 5-17: 500 nM, 200 nM, 100 nM, 50 nM, 20 nM, 10 nM, 5 nM, 2 nM, 1 nM, 500 pM, 200 pM, 100 pM, 50 pM, respectively.

FIG. 1d: Compound 3: Lane 5-17: 1 pM, 500 nM, 200 pM, 100 nM, 50 nM, 20 nM, 10 nM, 5 nM, 2 nM, 1 nM, 500 pM, 200 pM, 100 pM, respectively.

The analyzed binding site locations are indicated with square brackets along the left side of each autoradiogram.

### Example 5

### Protein Expression

Protein expression: Drosophila extradenticle (Exd) protein comprising the homeodomain and the extended fourth helix (residues 238-324), as well as ultrabithorax (Ubx) protein homeodomain (residues 233-313 of the Ubx isoform IVa) were expressed and purified after Passner & Aggarwal. The purified proteins Exd and Ubx were used for EMSA studies as described below.

### Example 6

### Gel Shift Studies (EMSA Studies)

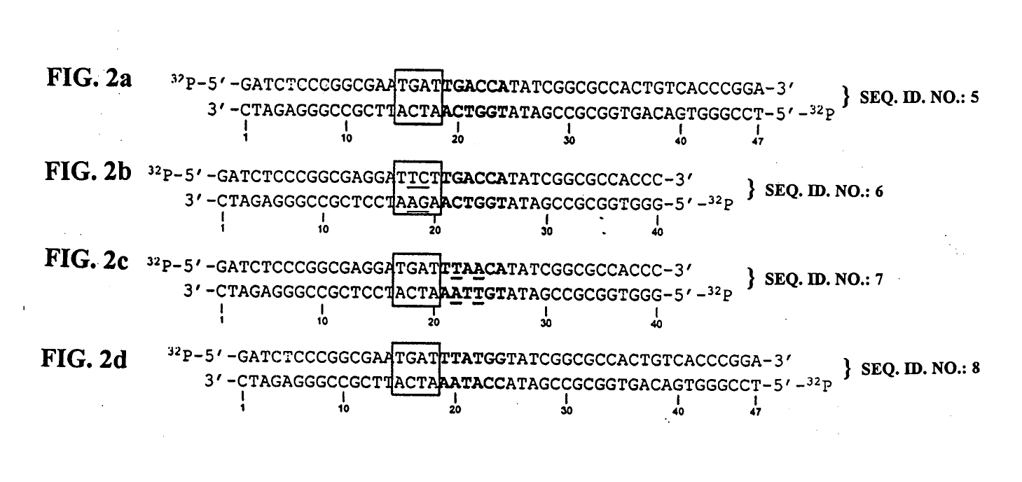

For the templates, the DNA oligonucleotides depicted in FIGS. 2a, 2b, 2c, and 2d were used, SEQ. ID NOS: 5, 6, 7, and 8, respectively. The DNA upper strand was annealed with the respective matching lower strand and both strands were 5′-labeled with γ-³²P-ATP and polynucleotide kinase, using standard procedures.

FIGS. 2a, 2b, 2c and 2d show depict the DNA duplexes used for the EMSA studies. The binding site for the Exd protein is marked by a box, the polyamide or Hox protein binding site is shown in boldface, FIG. 2a depicts the optimal template. FIG. 2b depicts a 2-bp mismatch in the Exd site. FIG. 2c depicts a 2-hp mismatch in the PA binding site. FIG. 2d depicts a composite Ubx-Exd binding site (see Passner et al. (1999) Nature, 397:714-719).

Gel-shift experiments: The master mix contained 50% BSA/50% glycerol, reaction buffer (150 mM potassium glutamate, 50 mM HEPES pH 7.0, 1 mM DTT, and 5′-end labeled DNA (³²P) The final concentrations in the samples were 100 ng/μL BSA and 10% glycerol. Polyamides were kept in subdued lighting whenever possible. Upon addition of the polyamide to 1 pM DNA, the samples were incubated at 25□ C. for 30 minutes in a 20 μL reaction. Next, Exd was added to the samples and incubated for 1 hour at 4° C. A 9% acrylamide/3% glycerol gel was pre-run for 15 min prior to loading. In each lane 15 μL of a 20 μL reaction were loaded while the gel was running to prevent the samples from being diluted. The gels were run at 4° C./185 V. Gels were dried, exposed to a phosphorimager screen, and visualized using a Molecular Dynamics phosphorimager.

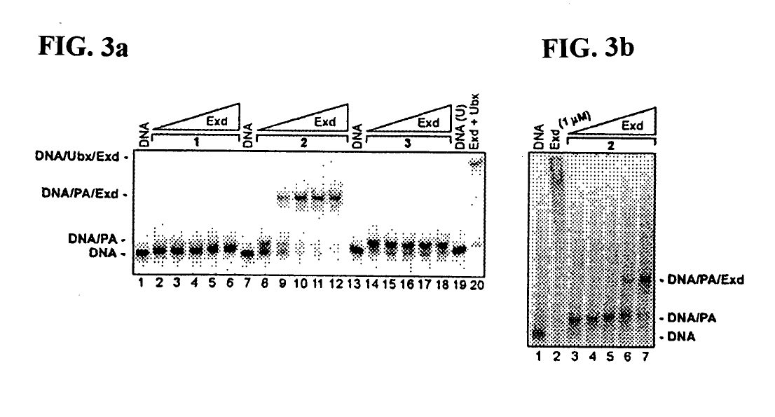

FIGS. 3a and 3b: EMSA studies with polyamides 1-3, Exd and Ubx. In FIG. 3a, each polyamide binds and decreases the mobility of free DNA (lanes 2-18). Compound 2 bearing the functional peptide 4 is capable of recruiting Exd to DNA (lanes 9-12) whereas 1 & 3 are not. In lanes 2-6,8-12, 14-18, Exd was added in following concentrations: 0, 3, 10, 30, 100 nM. Lanes 19 and 20 contained DNA bearing the Exd-Ubx binding site that was used in X-ray crystal structure determination (see methods above for sequence). In the reaction shown in lane 20, 275 nM Ubx and 30 nM Exd were incubated with DNA.

In FIG. 3b, multiple Exd molecules bind DNA at 1 μM concentration (lane 2), reactions in lanes 3-7 contain 50 nM PA 2 and increasing concentration of Exd (0, 0.3, 1, 3, and 10 nM in lanes 3-7, respectively).

Gel-shift studies with the Ubx protein were performed under identical buffer conditions using the duplex oligonucleotide listed in FIG. 2d. Ubx was added to the reaction mixture containing ³²P-endlabeled duplex DNA and incubated at 4° C. for 30 min. Subsequently Exd was added and the reaction was further incubated for 60 min at 4° C. The complexes were resolved under similar gel conditions as those described for DNA-polyamide-Exd complexes above. The K_{d} of Ubx for its cognate DNA was found to be 200±25 nM. Under saturating concentrations of Ubx (325 nM) the K_{d} of Exd for the binary [Ubx-DNA] complex was 2-3 fold larger than the affinity of Exd for the polyamide-DNA binary complex.

Discussion of Examples 1-6:

In Examples 1-6, a structure-based design was used to generate a composition of matter comprising a polyamide anchor moiety, a glycine linker moiety, and a polypeptide test compound. As shown in the above Examples, this approach that demonstrates that the test compound as presented the transcription factor, had functionality to recruit binding of the transcription factor to an isolated nucleic acid target. In the Examples, compound 2 displays a functional test compound and compound 3 displays a non-functional test compound attached to the PA-propylamine side chain via a glycine linker. As noted in the Example 3, the compounds were synthesized by solution-phase coupling of protected peptide acid fragments to the parent PA 1.

The DNA-binding properties of the compounds 1-3 were investigated by quantitative DNase 1 footprinting assays. The equilibrium binding constants of each of the compounds for a matched versus three single base pair mismatch sites is compiled in Table 1. The lower strand sequence is shown in the header. Mismatched base pairs are underlined. The residue under the YPWM peptide is in bold. Relative specificities are given in square brackets.

TABLE 1
Equilibrium Dissociation Constants Kd (nM) for 1, 2, and 3

| | TGGTCA | TGGCCA | TGGGCA | AGCTCA |
|---|---|---|---|---|
| Cmpd 1 | 0.048 ± 0.015 | 0.97 ± 0.41 [20] | 0.76 ± 0.39 [16] | 3.1 ± 0.9 [65] |
| Cmpd 2 | 5.8 ± 0.8 | 7.9 ± 1.6 [1.4] | .100 [17] | 6.4 ± 1.1 [1.1] |
| Cmpd 3 | 0.86 ± 0.32 | 12 ± 5 [14] | .100 [116] | 14 ± 5 [16] |

The conjugation of the peptides to the parent polyamide 1 leads to a reduction in binding affinity. But, the functional peptide sequence in 2 has a much greater influence on binding affinity and specificity than the mutant peptide in 3. Of particular note as shown in the Examples is the ability of the compounds 2 and 3 to discriminate between the CG and the GC mismatch base pair, a property not shown by the parent 1.

The ability of the compounds 1-3 to enhance Exd binding to its adjacent cognate site was tested using electrophoretic mobility shift assays (EMSA), Example 6. A 47-base-pair duplex DNA with one cognate site was incubated with saturating concentrations (50 nM) of each compound. As shown in FIG. 3a, polyamide-peptide” compounds according to the present invention bind DNA and slightly decrease its mobility (compare lanes 1 and 2 in FIG. 3a). In the presence of compound 2, Exd (residues 238-324) binds DNA with very high affinity. No binding was observed with the mutant peptide compound 3. The K_{d} of Exd for the DNA/compound 2 complex is 4.4±2 nM, 2-fold higher than that for DNA-Ubx complex. However, the affinity of Ubx for its DNA site is at least ˜40-fold lower than that of compound 2 for its respective site. Indeed, a 20-fold lower concentration of compound 2 is required to recruit Exd as compared to its natural Hox partner. Neither compound 3 nor 1 showed any ability to recruit Exd to its cognate site. At 1 μM, Exd binds DNA, but the mobility of the band suggests multiple Exd molecules bind to DNA nonspecifically. Thus, compound 2 improves the affinity of Exd for its cognate site by at least ˜200-fold, and far more importantly, it enhances specific binding of Exd to a target site.

To investigate the contribution of the peptide/Exd interaction to the binding site specificity, the polyamide-binding site on the DNA template was eliminated. As shown in FIG. 3b, compound 2 did not bind this mutated template even at 100 nM concentrations.

The Examples thus provide convincing evidence that: 1) the YPWM peptide contributes significantly to the cooperative interaction between a Hox protein and its partner on a DNA target; and 2) the present method is capable of evaluating and quantifying the nature of the cooperation. In summary, the Examples demonstrate that interactions between a nucleic acid-binding protein and its corresponding nucleic acid target can be evaluated using a composition of matter comprising a suitable DNA target having conjugated thereto a minor groove binding polyamide anchor/glycine linker/peptide test compound. The ability of compound 2 to recruit Exd more efficiently than its natural Hox protein partner illustrates that structure-based modular design is a valid strategy to test and evaluate both compound that modulate the action of artificial transcription factors, as well as a means to evaluate and test artificial transcription factors themselves.

By extension, the Examples indicate that this approach is not limited to generating test compounds or ATSs which mimic Hox factors. For example, joining two sequence-specific domains (one for the DNA target and one for a regulatory fact) via an intermediate linker will lead to cooperative protein/DNA dimerizers. Thus, a new class of small-molecule ATS can be obtained; compounds that function in concert with natural transcription factors.

### Example 7

### Effect of Varying Length of Linker Moiety

Eight compounds were synthesized each bearing the same polyamide and the FYPWM (SEQ. ID. NO: 13) penta-peptide test compound. Formula (a) depicts the anchor moiety as circles, and the linking moiety as X. Formula (b) depicts the positioning of the target nucleic acid, including the anchor moiety, linker, test compound and the Exd regulatory factor. Formula (c) shows the eight linking moieties used in this Example.

[image]

The propyl end of the linker projects off the N-methylpyrrole of the anchor moiety in each case. This arrangement is sufficient to project the test compound over the minor groove and position it adjacent to the major groove where Exd would bind. The eight varying linkers range from ˜33 Å in the case of the PEG linker to ˜2.5 Å in the case of the lysine linker. Linkers 1-5 and 8 bear an additional lysine residue at the C-terminus of their YPWM hook. This residue is often seen in hooks in various Hox proteins, in our case we treat it as an additional linker residue. The lysine also improves the solubility of these rather hydrophobic compounds. Compounds 6 and 7 do not bear the lysine and are less soluble. Each of the compounds were synthesized by solid phase methods, as described hereinabove. The polyamide was synthesized first, and then conjugated by conventional means to each of the eight linkers, which was then conjugated (by conventional means) to the peptide. Care was taken to ensure that the tyrosine residue was not racemized, and that the tryptophan was not oxidized. The compounds were confirmed by MALDI-TOF mass spectrometery (data not shown).

The DNA binding properties of each of the eight compounds were measured to determine if the linker altered their affinity or specificity for the target site. Two different assays were used to measure the affinity of the compounds for DNA. In the first approach, an electrophoretic mobility shift assay was performed wherein increasing amounts of each polyamide were incubated with a 50 bp duplex DNA molecule bearing a single optimal anchor moiety binding site. The polyamide-DNA complexes were resolved by electrophoresis on a 10% polyacrylamide gel. The incubation altered the mobility of the radioactively labeled DNA and an initial inspection of the gels suggested that at 20 nM each of the compounds saturated their binding site. DNAaseI footprinting was then performed to more precisely determine the affinity of the eight compounds for the binding site. The DNA fragment used in these assays also has sites that are closely related to the optimal polyamide binding site, varying only at one or two positions. Thus from a single footprinting reaction, the assay can determine subtle differences in the specificity of each compound for the optimal versus mismatch sites. The data generated for the eight linkers is presented in Table 2:

TABLE 2

| | Length | Extended | Jenks Δ | WMS ΔS |
|---|---|---|---|---|
| Linker No. | (Bonds) | Length Å | (cal/mol/° K.) | (cal/mol/° K.) |
| 1 | 27 | 32.59 | 121.5 + j | 43.9 + w |
| 2 | 9 | 11.14 | 40.5 + j | 15.5 + w |
| 3 | 7 | 8.65 | 31.5 + j | 12.4 + w |
| 4 | 6 | 7.42 | 27.0 + j | 10.8 + w |
| 5 | 5 | 6.14 | 22.5 + j | 9.27 + w |
| 6 | 4 | 4.97 | 18.0 + j | 7.64 + w |
| 7 | 3 | 3.81 | 13.5 + j | 6.09 + w |
| 8 | 2 | 2.46 | 9.0 + j | 4.54 + w |

Electrophoretic mobility shift assays (data not shown) indicated that each of the eight compounds was able to stabilize Exd binding to the adjacent cognate DNA site (TGAT). Neither the parent polyamide lacking the YPWM hook, nor the polyamide bearing an altered hook (FYPAAK) (SEQ. ID. NO: 14) was able to stabilize Exd binding (data not shown). The assays were performed with 50 nM of the conjugate pre-incubated with target DNA (thus to bind the anchor moiety to the target nucleic acid) followed by the addition and incubation with Exd. At 50 nM, each of the conjugates binds DNA stoichiometrically and the affinity of Exd can be readily monitored by the formation of ternary complex with increasing concentration of the conjugate. The data indicate that compounds incorporating linkers 7 and 8 (i.e., short linkers (<4 Å when fully extended), do not optimally position the test compound with respect to its hydrophobic docking site on the surface of Exd. The absence of the lysine residue in linker 6 did not appreciably alter the ability of the corresponding target nucleic acid to recruit Exd in comparison to the compound using linker 5 (which does bear the lysine residue and is roughly one angstrom longer).

Importantly, the data suggest that at 4° C., the linker range can vary from ˜5 Å to 33 Å (or more, likely up to about 50 Å) with a minimal cost to Exd binding. This result indicates the great utility in using longer linkers in initial screens to deliver test compounds whose interfacial recognition sites on the targeted regulatory factor have not been determined with any precision.

While binding at 4° C. shows a small effect of linker length on the ability of compound using linkers 1-6 to recruit Exd, it was likely the entropic penalty of bearing a larger linker would be more apparent at higher temperatures.

To determine the effects of longer linker on binding, the ability of compounds using linkers 1, 2 and 4 to recruit Exd at three different temperatures was investigated. From the experiments described in the previous paragraph, each of the compounds bearing linkers that project ˜33 Å, ˜11 Å and ˜7 Å (i.e., linkers 1, 2, and 4, respectively) was known to recruit Exd effectively at 4° C. However, the binding properties of these same compounds were found to be significantly different at room temperature (23° C.) and at physiological temperatures (37° C.). At 4° C., the three compounds recruit Exd with less than an order of magnitude difference in their apparent equilibrium dissociation constants (KD=0.6 nM for 1 vs. 0.08 nM for 4). However, linker 1, bearing a defined 28-atom polyethylene glycol linker, shows a dramatically reduced binding affinity at higher temperatures. Compound 2, with a shorter, 10-atom linker, is capable of recruiting Exd at room temperature but fails to do so effectively at physiological temperatures. The 7-atom linker is least responsive to the thermal variation with less than a 5-fold decrease in the ability to recruit Exd to DNA over a 33-degree change in temperature (4′-37° C.).

1. An in vitro method of evaluating one or more test compounds to identify test compounds that modulate binding of natural or artificial regulatory factors to corresponding single-, double-, or triple-stranded nucleic acid binding sites, the method comprising:
(a) providing an isolated nucleic acid target that defines at least one known or putative binding site for a regulatory factor, the nucleic acid target having conjugated or covalently bonded thereto, at a point proximate to, but not within, the binding site:
(i) an anchor moiety,
(ii) a linker moiety covalently bonded to the anchor moiety, and
(iii) a test compound bonded to the linker moiety; and then
(b) under transcription conditions, contacting in vitro the nucleic acid target of step (a) to a reagent mixture comprising one or more natural or artificial regulatory factors specific for the binding site defined in the nucleic acid target; and then
(c) determining whether binding of the regulatory factor to the binding site defined in the nucleic acid target is modulated by presence of the test compound.

2. The method of claim 1, wherein in step (a)(i), the anchor moiety comprises a polyamide or an intercalator.

3. The method of claim 1, wherein in step (a)(i), the anchor moiety comprises a moiety selected from the group consisting of a major-groove-binding/triple helix-forming oligonucleotide, a C₁₋₆ alkyl, a polycyclic aromatic hydrocarbon, a peptide nucleic acid, a polyamide, mitomycin C, cisplatin, and anthramycin.

4. The method of claim 1, wherein in step (a)(i), the anchor moiety is covalently bonded to the nucleic acid target.

5. The method of claim 1, wherein the isolated nucleic acid target defines one and only one known or putative binding site for a regulatory factor, and the nucleic acid target has conjugated or covalently bonded thereto one and only one anchor moiety.

6. The method of claim 5, wherein in step (a)(i), the anchor moiety comprises a moiety selected from the group consisting of a major-groove-binding/triple helix-forming oligonucleotide, a C₁₋₆ alkyl, a polycyclic aromatic hydrocarbon, an intercalator, a peptide nucleic acid, a polyamide, mitomycin C, cisplatin, and anthramycin.

7. The method of claim 5, wherein in step (a)(ii), the linker moiety comprises a bifunctional moiety selected from the group consisting of polypeptides, poly(ethyleneglycols), and C₁₋₆ alkylenyl, alkenyl, and alkynyl.

8. The method of claim 1, wherein in step (a)(ii), the linker moiety comprises a bifunctional moiety selected from the group consisting of polypeptides, poly(ethyleneglycols), and C₁₋₆ alkyl, alkene, and alkyne.

9. The method of claim 8, wherein in step (a)(i), the anchor moiety comprises a moiety selected from the group consisting of a major-groove-binding/triple helix-forming oligonucleotide, a C₁₋₆ alkyl, a polycyclic aromatic hydrocarbon, an intercalator, a peptide nucleic acid, a polyamide, mitomycin C, cisplatin, and anthramycin.

10. The method of claim 1, wherein in step (a) (ii), the linker moiety is an aptamer.

11. The method of claim 1, wherein in step (a)(ii), the linker moiety is at least 30 Å long.

12. The method of claim 1, wherein in step (a)(ii), the linker moiety is entropically destabilized such that entropy of the linker moiety confers conditional behavior upon the isolated nucleic acid target.

13. A method of evaluating one or more test compounds to identify test compounds that facilitate, recruit, or stabilize binding of natural transcription factors to corresponding single-, double-, or triple-stranded transcription factor binding sites on nucleic acid, the method comprising:
(a) providing an isolated nucleic acid target that defines at least one desired transcription factor binding site, the nucleic acid target having covalently bonded thereto, at a point proximate to, but not within, the transcription factor binding site:
(i) an anchor moiety,
(ii) a linker moiety covalently bonded to the anchor moiety, and
(iii) a test compound bonded to the linker moiety; and then
(b) under transcription conditions, contacting in vitro the nucleic acid target of step (a) to a reagent mixture comprising one or more natural transcription factors specific for the transcription factor binding site defined in the nucleic acid target; and then
(c) determining whether the test compound alters binding of the natural transcription factor to the nucleic acid target.

14. The method of claim 13, wherein the isolated nucleic acid target defines one and only one transcription factor binding site, and the nucleic acid target has covalently bonded thereto one and only one anchor moiety.

15. The method of claim 14, wherein in step (a)(i), the anchor moiety comprises a moiety selected from the group consisting of a major-groove-binding/triple helix-forming oligonucleotide, a C₁₋₆ alkyl, a polycyclic aromatic hydrocarbon, an intercalator, a peptide nucleic acid, a polyamide, mitomycin C, cisplatin, and anthramycin.

16. The method of claim 14, wherein in step step (a)(ii), the linker moiety comprises a bifunctional moiety selected from the group consisting of polypeptides, poly(ethyleneglycols), and C₁₋₆ alkylenyl, alkenyl, and alkynyl.

17. The method of claim 13, wherein in step step (a)(ii), the linker moiety comprises a bifunctional moiety selected from the group consisting of polypeptides, poly(ethyleneglycols), and C₁₋₆ alkyl, alkene, and alkyne.

18. The method of claim 17, wherein in step (a)(i), the anchor moiety comprises a moiety selected from the group consisting of a major-groove-binding/triple helix-forming oligonucleotide, a C₁₋₆ alkyl, a polycyclic aromatic hydrocarbon, an intercalator, a peptide nucleic acid, a polyamide, mitomycin C, cisplatin, and anthramycin.

19. The method of claim 13, wherein in step (a)(ii), the linker moiety is an aptamer.

20. The method of claim 13, wherein in step (a)(ii), the linker moiety is at least 30 Å long.

21. The method of claim 13, wherein in step (a)(ii), the linker moiety is entropically destabilized such that entropy of the linker moiety confers conditional behavior upon the isolated nucleic acid target.

22. A method of evaluating one or more test compounds to identify test compounds that facilitate, recruit, or stabilize binding of artificial transcription factors to corresponding single-, double-, or triple-stranded transcription factor binding sites on nucleic acid, the method comprising:
(a) providing an isolated nucleic acid target that defines at least one desired transcription factor binding site, the nucleic acid target having covalently bonded thereto, at a point proximate to, but not within, the transcription factor binding site:
(i) an anchor moiety,
(ii) a linker moiety covalently bonded to the anchor moiety, and
(iii) a test compound bonded to the linker moiety, wherein the test compound is known to modulate binding of natural transcription factors to the transcription factor binding site defined in the nucleic acid target; and then
(b) under transcription conditions, contacting in vitro the nucleic acid target of step (a) to a reagent mixture comprising one or more known or putative artificial transcription factors specific for the transcription factor binding site defined in the nucleic acid target; and then
(c) determining whether the test compound alters binding of the artificial transcription factor to the nucleic acid target.

23. The method of claim 22, wherein the isolated nucleic acid target defines one and only one transcription factor binding site, and the nucleic acid target has covalently bonded thereto one and only one anchor moiety.

24. The method of claim 23, wherein in step (a)(i), the anchor moiety comprises a moiety selected from the group consisting of a major-groove-binding/triple helix-forming oligonucleotide, a C₁₋₆ alkyl, a polycyclic aromatic hydrocarbon, an intercalator, a peptide nucleic acid, a polyamide, mitomycin C, cisplatin, and anthramycin.

25. The method of claim 23, wherein in step step (a)(ii), the linker moiety comprises a bifunctional moiety selected from the group consisting of polypeptides, poly(ethyleneglycols), and C₁₋₆ alkylenyl, alkenyl, and alkynyl.

26. The method of claim 22, wherein in step step (a)(ii), the linker moiety comprises a bifunctional moiety selected from the group consisting of polypeptides, poly(ethyleneglycols), and C₁₋₆ alkyl, alkene, and alkyne.

27. The method of claim 22, wherein in step (a)(i), the anchor moiety comprises a moiety selected from the group consisting of a major-groove-binding/triple helix-forming oligonucleotide, a C₁₋₆ alkyl, a polycyclic aromatic hydrocarbon, an intercalator, a peptide nucleic acid, a polyamide, mitomycin C, cisplatin, and anthramycin.

28. The method of claim 22, wherein in step (a)(ii), the linker moiety is an aptamer.

29. The method of claim 22, wherein in step (a)(ii), the linker moiety is at least 30 Å long.

30. The method of claim 22, wherein in step (a)(ii), the linker moiety is entropically destabilized such that entropy of the linker moiety confers conditional behavior upon the isolated nucleic acid target.

31. A composition of matter comprising an isolated nucleic acid target that defines a desired or putative binding site for a regulatory factor, the isolated nucleic acid target having covalently bonded thereto, at a point proximate to the binding site an anchor moiety, a linker moiety covalently bonded to the anchor moiety, and a test compound conjugated to the linker moiety.

32. The method of claim 31, wherein in step (a)(ii), the linker moiety is an aptamer.

33. The method of claim 31, wherein in step (a)(ii), the linker moiety is at least 30 Å long.

34. The method of claim 31, wherein in step (a)(ii), the linker moiety is entropically destabilized such that entropy of the linker moiety confers conditional behavior upon the isolated nucleic acid target.

35. A kit for testing a compound for its ability to modulate binding of a regulatory factor to a corresponding regulatory factor binding site on a nucleic acid, the kit comprising:
an isolated nucleic acid target that defines a regulatory factor binding site, the isolated nucleic acid target further comprising an anchor moiety covalently bonded thereto at a point proximate to the regulatory factor binding site, and a bifunctional linker moiety covalently bonded to the anchor moiety, wherein the bifunctional linker moiety comprises a free terminus that is dimensioned and configured to be conjugated to a compound to be tested; the isolated nucleic acid target being disposed in a suitable container, and
instructions for use of the kit.

36. The method of claim 35, wherein the bifunctional linker moiety is an aptamer.

37. The method of claim 35, wherein the bifunctional linker moiety is at least 30 Å long.

38. The method of claim 35, wherein the bifunctional linker moiety is entropically destabilized such that entropy of the linker moiety confers conditional behavior upon the isolated nucleic acid target.
